(19) United States
(12) Patent Application Publication
(10) Pub. No.: US 2014/0276483 A1
(43) Pub. Date: Sep. 18, 2014

(54) TRANSDERMAL METHYLPHENIDATE COMPOSITIONS WITH ACRYLIC BLOCK COPOLYMERS

(71) Applicant: Noven Pharmaceuticals, Inc., Miami, FL (US)

(72) Inventors: Jun Liao, Miami, FL (US); Puchun Liu, Miami, FL (US); Steven Dinh, Miami, FL (US)

(73) Assignee: Noven Pharmaceuticals, Inc., Miami, FL (US)

(21) Appl. No.: 14/208,398

(22) Filed: Mar. 13, 2014

Related U.S. Application Data
(60) Provisional application No. 61/785,325, filed on Mar. 14, 2013.

(51) Int. Cl. A61K 47/32; A61K 9/70; A61K 31/4458

(52) U.S. Cl. CPC A61K 47/32; A61K 31/4458; A61K 9/7007
USPC 604/307; 514/317; 424/443; 427/2.31

(57) ABSTRACT

Described herein are transdermal compositions comprising methylphenidate in a polymer matrix comprising a non-reactive random acrylic polymer and an acrylic block copolymer. Method of making such compositions, and therapeutic methods using them also are disclosed.

### CROSS-REFERENCE TO RELATED APPLICATIONS

This application claims the benefit under 35 U.S.C. §119(e) to U.S. provisional application 61/785,325, filed Mar. 14, 2013, the contents of which are incorporated here by reference in their entirety.

### FIELD

The present invention relates generally to the transdermal delivery of methylphenidate, and to compositions and methods for transdermally delivering methylphenidate, such as may be desired for treating Attention Deficit Disorder (ADD) and/or Attention Deficit/Hyperactivity Disorder (ADHD), postural orthostatic tachycardia syndrome, and narcolepsy.

### BACKGROUND

Many factors influence the design and performance of transdermal drug delivery compositions. These include the individual drugs themselves, the physical and chemical characteristics of the compositions' components and their performance and behavior relative to other components, external and environmental conditions during manufacturing and storage, properties of the application site, the desired rate of drug delivery and therapeutic onset, the desired drug delivery profile, and the intended duration of delivery, among others.

Methylphenidate (MPH) is the most commonly prescribed psychostimulant drug approved for treatment of attention-deficit hyperactivity disorder (ADHD), postural orthostatic tachycardia syndrome, and narcolepsy. It can be administered through different routes, including oral, sublingual, transdermal, intravenous and nasal.

Daytrana® (methylpheidate transdermal system) is the only commercially available transdermal product for methylphenidate. It is available in four dosage strengths (10, 15, 20 and 30 mg/day) and is applied for 9 hours using patch sizes correlated with the dose (12.5, 18.75, 25 and 37.5 cm², respectively). Daytrana® is a drug-in-adhesive matrix system that is composed of methylphenidate in a polymer matrix comprised of an acrylic pressure-sensitive adhesive and a silicone pressure-sensitive adhesive. Daytrana® effectively delivers methylphenidate with satisfactory skin adhesion; however, the peel force from the release liner of the patch increases over time. This becomes a serious issue, as patients may encounter difficulties or be unable to remove the release liner as required for use. Thus, there remains a need for transdermal compositions comprising methylphenidate that exhibit suitable physical and pharmacokinetic properties.

### SUMMARY OF THE INVENTION

Described herein are compositions for the transdermal delivery of methylphenidate in the form of a flexible finite system for topical application, comprising a polymer matrix comprising methylphenidate or a pharmaceutically acceptable salt or prodrug thereof, wherein the polymer matrix comprises a non-reactive random acrylic polymer and an acrylic block copolymer.

In some embodiments, the non-reactive random acrylic polymer is made from one or more monomers selected from the group consisting of methyl acrylate, methyl methacrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, and octyl acrylamide. In some embodiments, the polymer matrix comprises from about 10% to about 90% by weight non-reactive random acrylic polymer, or from about 30% to about 50% by weight non-reactive random acrylic polymer.

In some embodiments, the acrylic block copolymer is made from one or more monomers selected from the group consisting of methyl acrylate, methyl methacrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate. 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, and octyl acrylamide. In some embodiments, the acrylic block copolymer comprises one or more of poly(butyl acrylate), poly(methyl methacrylate), methyl methacrylate/butyl acrylate (MMA/BA) blocks, and methyl methacrylate/butyl acrylate/alpha methyl styrene/polypropylene glycol (MMA/BA/AMS/PPG) blocks. In some embodiments, the polymer matrix comprises from about 1% to about 70% by weight acrylic block copolymer, or from about 10% to about 60% by weight acrylic block copolymer.

In any embodiments, the methylphenidate may be methylphenidate free base. In some embodiments, the polymer matrix comprises from about 10% to about 50% by weight methylphenidate free base, or from about 20% to about 40% by weight methylphenidate free base.

In some embodiments, the composition is capable of delivering methylphenidate over a period of time of from about 6 to about 10 hours, such as a period of time of about 9 hours.

In any embodiments, the composition may further comprise a backing layer and/or a release liner.

In some embodiments, the composition is for the transdermal delivery of methylphenidate.

Also provided are methods for the transdermal delivery of methylphenidate, comprising topically applying a composition as described herein to the skin or mucosa of a subject in need thereof.

Also provided arc methods of manufacturing a composition for the transdermal delivery of methylphenidate in the form of a flexible finite system for topical application, comprising forming a polymer matrix blend comprising a non-reactive random acrylic polymer, an acrylic block copolymer, and methylphenidate or pharmaceutically acceptable salt or prodrug thereof in a solvent, applying the polymer matrix blend to a support layer, and removing any remaining solvent.

### BRIEF DESCRIPTION OF THE DRAWINGS

FIG. 1 shows the in vitro flux data (flux, μg/cm²/hr) for the compositions of Example 1.

FIG. 2 shows the in vitro flux data (flux, μg/cm²/hr) for the compositions of Example 2.

FIGS. 3 and 4 show the in vitro flux data (flux, μg/cm²/hr) for the compositions of Examples 3 and 4 (for each skin sample).

### DETAILED DESCRIPTION

Described herein are transdermal compositions comprising methylphenidate in a polymer matrix comprising a non-reactive acrylic polymer and an acrylic block copolymer. The compositions include exhibit suitable physical and pharmacokinetic properties, and do not suffer from the release liner peel problems associated with Daytrana®.

Definitions

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrase “substantially free” as used herein means that the described composition (e.g., polymer matrix, etc.) comprises less than about 5%, less than about 3%, or less than about 1% by weight, based on the total weight of the composition at issue, of the excluded component(s).

As used herein “subject” denotes any mammal in need of drug therapy, including humans. For example, a subject may be suffering from or at risk of developing a condition that can be treated or prevented with methylphenidate (such as ADD or ADHD, postural orthostatic tachycardia syndrome, or narcolepsy), or may be taking methylphenidate for health maintenance purposes.

As used herein, the terms “topical” and “topically” mean application to a skin or mucosal surface of a mammal, while the terms “transdermal” and “transdermal” connote passage through the skin or mucosa (including oral, buccal, nasal, rectal and vaginal mucosa), into systemic circulation. Thus, the compositions described herein may be applied topically to a subject achieve transdermal delivery of methylphenidate.

As used herein, the phrases ⁻therapeutically effective amount” and “therapeutic level” mean that drug dosage or plasma concentration in a subject, respectively, that provides the specific pharmacological effect for which the drug is administered in a subject in need of such treatment. It is emphasized that a therapeutically effective amount or therapeutic level of a drug will not always be effective in treating the conditions/diseases described herein, even though such dosage is deemed to be a therapeutically effective amount by those of skill in the art. For convenience only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease.

As used herein, “active surface area” means the surface area of the drug-containing polymer matrix of the transdermal drug delivery system.

The compositions described herein are in a “flexible, finite form.” As used herein, the phrase “flexible, finite form” means a substantially solid form capable of conforming to a surface with which it comes into contact, and capable of maintaining contact so as to facilitate topical application. Such systems in general are known in the art and commercially available, such as transdermal drug delivery patches. The compositions comprise a drug-containing polymer matrix that releases an active agent (such as methylphenidate) upon application to the skin (or any other surface noted above). In some embodiments, the composition in flexible, finite form may include a backing layer and/or a release liner layer in addition to a drug-containing polymer matrix layer.

As used herein, “drug-containing polymer matrix” refers to a polymer composition which contains one or more drugs, such as methylphenidate, and a polymer, such as a pressure-sensitive adhesive polymer or a bioadhesive polymer. A polymer is an “adhesive” or “bioadhesive” if it has the properties of adhesiveness per se. Other polymers can function as an adhesive or bioadhesive by the addition of tackifiers, plasticizers, crosslinking agents, skin permeation enhancers, or other excipients. Thus, in some embodiments, the polymer optionally comprises tackifiers, plasticizers, crosslinking agents or other additives known in the art.

As used herein, the term “pressure-sensitive adhesive” refers to a viscoelastic material which adheres instantaneously to most substrates with the application of very slight pressure and remains permanently tacky. As noted above, a polymer is a pressure-sensitive adhesive polymer if it has the properties of a pressure-sensitive adhesive per se. Other polymers may function as a pressure-sensitive adhesive by admixture with tackifiers, plasticizers or other additives. The term pressure-sensitive adhesive also includes mixtures of different polymers.

As used herein, the term “non-reactive component” identifies components that do not contain functional groups with active hydrogen atoms or functional groups with hydrogen atoms available for chemical reaction or interaction with methylphenidate, such as, for example, carboxyl, hydroxyl, amine, thiol, silanol, or epoxy groups. As used herein, non-reactive components may include amide group-containing monomers (e.g., components with amido groups).

In some embodiments, the polymer matrix is a pressure-sensitive adhesive at room temperature and exhibits desirable physical properties, such as good adherence to skin, ability to be peeled or otherwise removed without substantial trauma to the skin, retention of tack with aging, etc.

Methylphenidate

Methylphenidate (a-phenyl-2-piperidineacetic acid methyl ester) is a chiral drug. While commercially available methylphenidate products (such as the oral product Ritalin® tablets and the transdermal product Daytrana® patch) include a 50:50 (racemic) mixture of d- and l-threo-methylphenidate, it is believed that the d-threo-methylphenidate isomer has greater pharmacological activity. The compositions described herein may be formulated with any isomer of methylphenidate, although compositions comprising a racemic mixture of d- and l-threo-methylphenidate, or comprising primarily the d-threo-methylphenidate isomer may be most commercially relevant.

The compositions described herein may be formulated with methylphenidate free base (“methylphenidate base”), any pharmaceutically acceptable salt thereof, or mixtures thereof. Exemplary suitable pharmaceutically acceptable salts of methylphenidate are salts of weak inorganic and organic acids, and quaternary ammonium salts. These include without limitation, salts with acids such as sulfuric, phosphoric, hydrochloric, hydrobromic, hydriodic, sulfamic, citric, lactic, maleic, malic, succinic, tartaric, cinnamic, acetic, benzoic, gluconic, or ascorbic acid, or quaternary ammonium salts with organic esters of sulfuric, hydrohalic, or aromatic sulfonic acids, such as methyl chloride, methyl bromide, ethyl chloride, propyl chloride, butyl chloride, isobutyl chloride, benzylchloride, benzyl bromide, phenethyl bromide, naphthymethyl chloride, dimethyl sulfate, methyl benzenesulfonate, ethyl toluenesulfonate, ethylene chlorohydrin, propylene chlorobydrin, allyl bromide, methylallyl bromide or crotyl bromide esters.

Methylphenidate, including methylphenidate base in particular, has a secondary amine moiety and a methyl ester moiety, and is unstable and undergoes degradation in the presence of reactive functional groups, such as active hydrogen atoms or functional groups with hydrogen atoms available for chemical reaction or interaction with methylphenidate, such as, for example, carboxyl, hydroxyl, amine, thiol, silanol or epoxy groups, which may be present in polymers, enhancers, excipients and other components that typically may be used in transdermal compositions. Major degradants of methylphenidate include ritalinic acid and erythol isomer, whose concentrations increase significantly with increasing amounts (by weight) of functional groups. Such degradation can greatly reduce the amount of the active species present in a composition after storage, thus reducing the amount of active methylphenidate available for drug delivery. Thus, in some embodiments, the compositions described herein are formulated without components that have such functional groups. That is, in some embodiments, the compositions described herein are formulated only with non-reactive components as defined above and discussed in more detail below.

The polymer matrix compositions described herein include a therapeutically effective amount of methylphenidate or pharmaceutically acceptable salt thereof. Generally, the amount of methylphenidate is from about 1% to about 50%, including from about 10% to about 50%, such as from about 20% to about 40% by weight, including about 20%, about 25%, about 30%, about 35%, about 40%, about 45%, and about 50% by weight, based on the total dry weight of the polymer matrix.

In accordance with any of the embodiments described herein, the composition may include from about 20 to about 225 mg per unit of methylphenidate base or an equivalent amount of a pharmaceutically acceptable salt thereof.

Polymer Matrix

As noted above, the compositions described herein comprise methylphenidate formulated in a polymer matrix (e.g., a drug-in-adhesive polymer matrix). In some embodiments, the polymer matrix comprises a non-reactive acrylic polymer and an acrylic block copolymer (ABC). Compositions as described herein have an improved stability profile as compared to the commercial Daytrana® product, and do not suffer from the release liner peel problem. Moreover, in some embodiments, compositions as described herein achieve significantly higher flux at higher drug loading, permitting the use of smaller patches (e.g., systems with a smaller active surface area) to deliver same amount of the drug as Daytrana®.

Transdermal methylphenidate compositions comprising acrylic polymers have been described; nevertheless, difficulties remain. For example, because methylphenidate has a relatively high solubility in acrylic pressure-sensitive adhesives, high drug loading (e.g., 20% by weight drug) generally is required to achieve a drug flux that is comparable to the commercial Daytrana® product. However, methylphenidate also tends to plasticize acrylic polymer matrix compositions, and the plasticizing effect increases significantly with increased drug loading (e.g., increased drug concentration), leading to cold flow. Thus, when formulating methylphenidate in an acrylic polymer composition, the choice usually is made between designing a composition that avoids cold flow problems but achieves a much lower flux than the commercial Daytrana® product, due to relatively low drug loading, and a composition that achieves better drug flux but exhibits cold flow, due to higher drug loading. The present invention addresses this problem by providing a polymer matrix that includes a non-functional acrylic polymer and an acrylic block copolymer. While not wanting to be bound by any theory, it is believed that the ABC improves the physical properties of the matrix, such as increasing the cohesion properties of the matrix, permitting relatively high drug loading without encountering cold flow problem (e.g., oozing).

Non-Reactive Random Acrylic Polymers

As noted above, the compositions described herein include a polymer matrix comprising a non-reactive random acrylic polymer, such as one or more non-reactive pressure-sensitive adhesive random acrylic polymers. As used herein, the term “random” as modifying “polymer” is used in contrast to block copolymers, and refers to acrylic polymers that have a random arrangement of monomer units.

As used herein, “non-reactive acrylic polymer” includes any acrylic polymers that do not include functional groups that are reactive with methylphenidate, as discussed above. Such acrylic polymers include any acrylic-type of polymers comprised of monomers that do not include functional groups reactive with methylphenidate, such as acid-functional or hydroxy-functional groups, as discussed above. Examples of suitable non-reactive acrylic polymers include those formed from acrylic esters copolymerized with other monomers that do not include groups that are reactive with methylphenidate, and include homopolymers, copolymers, terpolymers, etc., of esters or amides of acrylic-type carboxylic acids. In some embodiments, the acrylic polymer comprises one or more non-reactive acrylic polymers with a random, block, graft and/or hybrid structure.

Suitable acrylic polymers can be obtained commercially or by polymerizing or copolymerizing suitable monomers such as acrylic monomers and other polymerizable monomers. Acrylate monomers which can be used include alkyl acrylates and alkyl methacrylates, such as methacrylic acid, methyl acrylate, methyl methacrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, and tridecyl methacrylate, and amide-group containing-monomers, such as octyl acrylamide. In specific embodiments, the non-reactive acrylic polymer includes methyl acrylate monomers and 2-ethylhexyl acrylate monomers. In other specific embodiments the non-reactive acrylic polymer includes methyl methacrylate monomers, 2-ethylhexyl acrylate monomers, butyl acrylate monomers and amide-group containing monomers.

Suitable non-reactive random acrylic polymers which are commercially available include those sold by Henkel North America under the Duro-Tak® brand name such as Duro-Tak® 87-900A, 87-901A, 87-9085, 87-9088, 87-9301A, and by Cytec Industries Inc. under under the Gelva® GMS brand name, such as Gelva® GMS 3067, 3071, 3083, 3087 and 3235. Other suitable acrylic polymers are known in the art. See, e.g., the non-reactive acrylic polymers described in Satas, “Acrylic Adhesives, HANDBOOK OF PRESSURE-SENSITIVE ADHESIVE TECHNOLOGY, 2nd ed., pp. 396-456 (D. Satas, ed.), Van Nostrand Reinhold, N. Y. (1989); “Acrylic and Methacrylic Ester Polymers,” POLYMER SCIENCE AND ENGINEERING, Vol. 1, 2nd ed., pp 234-268, John Wiley & Sons, (1984).

In any embodiments, the random acrylic polymer may comprise a mixture of two or more random acrylic polymers in any relative amounts. In some embodiments, the type(s) and amount(s) of non-reactive random acrylic polymer(s) is selected to achieve a composition with desired physical or pharmacokinetic properties. For example, as noted above, the type and amount of random acrylic polymer can impact the solubility of methylphenidate in the polymer matrix, which in turn can impact drug loading and pharmacokinetics, such as the rate and duration of drug delivery.

Generally, the polymer matrix includes from about 10% to about 90% by weight non-reactive random acrylic polymer, including from about 20% to about 80%, such as from about 30% to about 50% by weight, including about 10%, about 20%, about 25%, about 30%, about 32.5%, about 35%, about 40%, about 45%, and about 50% by weight, based on the total dry weight of the polymer matrix.

Acr lie Block Copolymers

As noted above, in addition to a non-reactive random acrylic polymer, the polymer matrix comprises one or more acrylic block copolymers (ABCs), such as one or more pressure-sensitive adhesive acrylic block copolymers.

Suitable ABCs can be made from acrylate monomers such as alkyl acrylates and alkyl methacrylates, such as methyl acrylate, methyl methacrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate. 2-ethylhexyl acrylate, 2-ethylhexyl methacrylate, decyl acrylate, decyl methacrylate. dodecyl acrylate, dodecyl methacrylate, tridecyl acrylate, tridecyl methacrylate, and octyl acrylamide. In some embodiments, the ABC is comprised of methyl methacrylate/butyl acrylate (MMA/BA) blocks. In some embodiments, the ABC is comprised of methyl methacrylate/butyl acrylate/alpha methyl styrene/polypropylene glycol (MMA/BA/AMS/PPG) blocks. In some embodiments, the ABC comprises poly(butyl acrylate) and/or poly(methyl methacrylate). Suitable acrylic block copolymers are available commercially, such as from Henkel, e.g., Duro-Tak® 87-9900.

In any embodiments, the ABC polymer may comprise a mixture of two or more ABCs in any relative amounts. For example, the type(s) and amount(s) of ABCs can be selected and controlled to select and control the physical and/or pharmacokinetic properties of the polymer matrix. For example, in some embodiments, the ABC includes poly(butyl acrylate) and poly(methyl methacrylate).

The ABC may be present in any amount. In some embodiments, the ABC comprises from about 1% to about 70% by weight of the polymer matrix, including from about 10% to about 60%, including from about 30% to about 50%, such as about 35%, about 40%, about 45%, and about 50%.

In some embodiments, the polymer matrix is substantially free of silicone polymers. In some embodiments, the polymer matrix is free of silicone polymers. By “free of silicone polymers” is meant that the composition is formulated without silicone polymers, such that at most only trace amounts are present as impurities or contaminants.

Other Components

The polymer matrix of the compositions described herein optionally may further comprise other components typically used in a transdermal drug delivery composition, such as antioxidants, skin permeation enhancers, tackifiers, plasticizers, crosslinking agents, or other excipients known in the art. In some embodiments, any such components are non-reactive components, as discussed above.

Antioxidants

In some embodiments, the polymer matrix includes an antioxidant. In some embodiments, the antioxidant is butylhydroxytoluene (BHT) and/or butylhydroxyanisole (BHA). In other embodiments, the antioxidant is, additionally or alternatively, tertiary-butylhydroquinone (TBHQ), alpha tocopherol, ascorbic-acid, ascorbyl palmitate, propyl gallate, fumaric acid, malic acid, sodium ascorbate, sodium metabisulfite, and the like. In some embodiments, the antioxidant is a non-reactive component as discussed above. In specific embodiments, the antioxidant (or combinations thereof) are used in a total amount of from about 0 to about 1.0% by weight, including from about .1 to about 1.0% by weight, such as about 0.1% by weight, about 0.25% by weight, and about 0.5% by weight, based on the dry weight of the polymer matrix.

Penetration Enhancers

Although methylphenidate does not generally require a penetration enhancer, in some embodiments, the polymer matrix comprises a penetration enhancer. A “penetration enhancer” is an agent known to accelerate the delivery of the drug through the skin. These agents also have been referred to as accelerants, adjuvants, and sorption promoters, and are collectively referred to herein as “enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer. In some embodiments, the penetration enhancer is a non-reactive component as discussed above.

Illustrative penetration enhancers include but are not limited to polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethyidecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

In some embodiments, the polymer matrix does not comprise a penetration enhancer.

When present, a penetration enhancer typically is used in an amount up to about 30% by dry weight of the polymer matrix, including up to 30% by weight, up to about 20% by weight, including 20% by weight, or up to about 10% by weight, up to 10% by weight, or up to 5% by weight, including up to 5% by weight, based on the dry weight of the polymer matrix.

Tackifying Agents

In some embodiments, the polymer matrix comprises one or more tackifying agents, such as aliphatic hydrocarbons, mixed aliphatic and aromatic hydrocarbons, aromatic hydrocarbons, substituted aromatic hydrocarbons, hydrogenated esters, polyterpenes, silicone fluid, mineral oil and hydrogenated wood rosins. In some embodiments, the polymer matrix includes one or more tackifying agents selected from rosin esters, aliphatic hydrocarbon resins, aromatic hydrocarbon resins, terpene resins, polybutene, and hydrogenated polybutene. In specific embodiments, the polymer matrix includes one or more C5 to C9 hydrogenated hydrocarbon resins (HHR).

Other Excipients

In some embodiments, the polymer matrix includes one or more thickeners, fillers, and/or other additives or components known for use in transdermal drug delivery systems.

For example, in some embodiments, the polymer matrix includes one or more of soluble and insoluble polyvinylpyrrolidones (PVP), ethylene-vinyl acetate copolymers, cellulose derivatives, and silicone dioxide (SiO₂), and other components.

In some embodiments, the polymer matrix includes one or more binders, such as lecithin, which “bind” the other ingredients; one or more rheological agents (thickeners) containing silicone, such as fumed silica, reagent grade sand, precipitated silica, amorphous silica, colloidal silicon dioxide, fused silica, silica gel, quartz and particulate siliceous materials commercially available as Syloid®, Cabosil®, Aerosil®, and Whitelite®, such as for enhancing the uniform consistency or continuous phase of the composition or coating.

Other additives and excipients include diluents, stabilizers, fillers, clays, buffering agents, biocides, humectants, anti-irritants, preservatives, plasticizing agents, cross-linking agents, flavoring agents, colorants, pigments and the like.

These substances can be present in any amount sufficient to impart the desired properties to the composition, and are typically used in amounts totaling up to 50%, including from about 0.1% to about 30%, by weight based on the dry weight of the polymer matrix. As noted above, in some embodiments, any such components are non-reactive components.

Backing Layer

Any of the compositions described herein may include a drug impermeable backing layer or film, adjacent one face of the polymer matrix. (By “impermeable” to the drug is meant that no substantial amount of drug loss through the backing layer is observed.) When present, the backing layer protects the polymer matrix from the environment and prevents loss of the drug and/or release of other components to the environment during use. Materials suitable for use as backing layers are well-known known in the art and can comprise films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers. Suitable backing materials include commercially available backings films, such as breathable backings such as 3M CoTran™ backings which feature low moisture vapor transmission rate and high oxygen transmission, and non-breathable polyester-based laminate backings such as 3M Scotchpak® backings (3M. St. Paul, Minn.).

Release Liner

Any of the compositions described herein may include a release liner, typically located adjacent the opposite face of the system as compared to the backing layer. When present, the release liner is removed from the system prior to use to expose the polymer matrix layer prior to topical application. Materials suitable for use as release liners are well-known known in the art and commercially available, and include silicone-coated polyethylene, polypropylene, polyester, and polystyrene release liners sold under the PRIMELINER™ brand as supplied by Loparex LLC (Cary, N.C.) and 3M Scotchpak™ fluoropolymer-coated polyester release liners supplied by 3M (St. Paul, Minn.).

Manufacturing Methods

The compositions described here can be prepared by methods known in the art, such as blending (mixing) the polymer(s), tackifier(s) and, as needed, other excipients with an appropriate amount of the drug in the presence of an appropriate solvent, such as a volatile organic solvent, casting the wet blend onto a release liner, followed by evaporation of the volatile solvent(s) at appropriate drying conditions, laminating the dried drug-in-adhesive layer on the release liner onto a backing film.

In accordance with any of the embodiments of the compositions described herein, the coat weight of the polymer matrix can be, in some embodiments, from about 3 mg/cm² to about 20 mg/cm², based on the active surface area of the polymer matrix. Exemplary coat weights include about 3 mg/cm², about 4 mg/cm², about 5 mg/cm², about 5.5 mg/cm², about 6 mg/cm², about 6.5 mg/cm², about 7 mg/cm², about 7.5 mg/cm², about 8 mg/cm², about 8.5 mg/cm², about 9 mg/cm², about 9.5 mg/cm², about 10 mg/cm², about 12 mg/cm², about 15 mg/cm², about 17 mg/cm², and about 20 mg/cm².

In accordance with any of the embodiments of the compositions described herein, the methylphenidate can be present, in some embodiments, in an amount from about 0.5 mg/cm² to about 3 mg/cm², including from about 1.5 mg/cm² to about 4 mg/cm², based on the active surface area of the of the polymer matrix. Exemplary amounts include about 0.5 mg/cm², about 0.8 mg/cm², about 1 mg/cm², about 1.2 mg/cm², about 1.4 mg/cm², about 1.6 mg/cm², about 1.7 mg/cm², about 1.8 mg/cm², about 2.0 mg/cm², about 2.2 mg/cm², about 2.4 mg/cm², about 2.6 mg/cm², about 2.8 mg/cm², and about 3.0 mg/cm², about 3.7 mg/cm², about 4.5 mg/cm² and about 5.0 mg/cm².

An exemplary general method for preparing a unit final product of a composition as described herein in a flexible, finite form, is as follows:

1. Appropriate amounts of one or more polymers, solvent(s) and/or co-solvent(s), and optional excipient(s) are combined and thoroughly mixed together in a vessel.

2. The methylphenidate is added to the mixture and agitation is carried out until the drug is uniformly mixed therein.

3. The composition is transferred to a coating operation where it is coated onto a release liner at a controlled specified thickness. The coated composition is then passed through an oven in order to drive off all volatile processing solvents.

4. The composition coated on the release liner is then brought into contact with a backing layer and wound into rolls.

5. Appropriate size and shape delivery systems are die-cut from the roll material and then pouched.

The order of steps, the amount of the ingredients, and the amount and time of agitation or mixing may be important process variables which will depend on the specific polymers, active agents, solvents and/or co-solvents, and optional excipients used in the composition, but these factors can be adjusted by those skilled in the art. The order in which each method step is performed can be changed if needed without detracting from the invention.

In accordance with any of the embodiments of compositions described herein, the size of the final product is, in some embodiments, in the range of from about 2 cm² to about 60 cm², including from about 15 cm² to about 30 cm², including 12.5 cm², 14.5 cm², 15 cm², 18.75 cm², 22.5 cm², 25 cm², 30 cm², 37.5 cm², and 45 cm².

Methods of Use

The compositions described herein are useful in methods for the transdermal delivery of methylphenidate, including in methods for treating attention deficit disorder and/or attention deficit/hyperactivity disorder, postural orthostatic tachycardia syndrome, and narcolepsy. In such embodiments, a composition comprising a therapeutically effective amount of methylphenidate as described herein is topically applied to a subject in need thereof.

In some embodiments, the compositions achieve transdermal delivery of methylphenidate over a period of time of at least about 8 hours, including a period of time of at least about 8 hours to at least about 12 hours. In some embodiments, the compositions achieve transdermal delivery of methylphenidate over a period of time of about 8 hours, about 9 hours, about 10 hours, or longer, including up to and including about 24 hours. In some embodiments, the compositions are formulated for daily application.

The compositions described herein achieve a transdermal flux of methylphenidate (or a pharmaceutically acceptable salt thereof) that is sufficient to have a therapeutic effect. As used herein, “flux” (also called “permeation rate”) is defined as the absorption of a drug through skin or mucosal tissue, and is described by Pick's first law of diffusion:

J=−D(dCm/dx)

where J is the flux in g/cm²/sec, D is the diffusion coefficient of the drug through the skin or mucosa in cm²/sec and dCm/dx is the concentration gradient of the drug across the skin or mucosa.

In accordance with other embodiments, there are provided compositions as described herein for use in the transdermal delivery of methylphenidate, such as for use by topically application to the skin or mucosa of a subject in need thereof.

The following specific examples are included as illustrative of the compositions described herein. These example are in no way intended to limit the scope of the invention. Other aspects of the invention will be apparent to those skilled in the art to which the invention pertains.

### EXAMPLE 1

Various polymer matrix compositions were prepared as described below, and applied at a coat weight of 6.6 mg/cm² to a backing (e.g. a polyester/ethylene vinyl acetate film, such as ScotchPak® 9732) and a release liner (e.g., a silicone- or fluoropolymer-coated polyester film). Drug flux over 9 hours was assessed in vitro using human cadaver skin (n=3):

| | | 9 hour | |
|---|---|---|---|
| | | Flux | Flux |
| Formula | Composition | (ug/cm2/h) | Ratio |
| Daytrana ® | Daytrana ® Product | 19.6 | 1 |
| 42660 (♦) | | | |
| 1-1 (▪) | 20% Methylphenidate | 15.1 | 0.77 |
| | 40% GMS ® 3087 | | |
| | 40% Duro-Tak ® 87-9900 | | |
| 1-2 (▴) | 20% Methylphenidate | 16.9 | 0.86 |
| | 40% Duro-Tak ® 87-900A | | |
| | 40% Duro-Tak ® 87-9900 | | |
| 1-3 (Δ) | 20% Methylphenidate | 13.4 | 0.68 |
| | 30% Duro-Tak ® 87-900A | | |
| | 50% Duro-Tak ® 87-9900 | | |
| 1-4 (∘) | 25% Methylphenidate | 16.3 | 0.83 |
| | 40% GMS ® 3087 | | |
| | 35% Duro-Tak ® 87-9900 | | |
| 1-5 () | 25% Methylphenidate | 19.4 | 0.99 |
| | 35% Duro-Tak ® 87-900A | | |
| | 40% Duro-Tak ® 87-9900 | | |

Results are shown in FIG. 1 (flux/avg. permeation rate, μg/cm²/hr). The results show that in vitro flux increases with increasing methylphenidate loading. Compositions with 25% by weight methylphenidate achieved drug flux comparable to the Daytrana.® product.

The peel properties of the compositions from a release liner were studied over 16 weeks at ambient conditions. The compositions were packaged in a package system comparable to that used for the Daytrana.® product e.g., in an inner pouchstock material provided in an outer package in a polypropylene tray with a silica gel desiccant. Results (Avg. Peel from Release Liner (g/0.5″) (n=3) are shown below:

| | | 4 | 8 | 12 | 16 |
|---|---|---|---|---|---|
| Formula | Initial | week | week | week | week |
| 1-1 | 1.8 | 1.6 | 1.3 | 0.5 | 1.1 |
| 1-2 | 2.6 | 2.5 | 1 | 2 | 1.9 |
| 1-3 | 1.3 | 1.5 | 0.6 | 1 | 1.2 |
| 1-4 | 0.9 | N/A | 1.3 | 1.4 | 1.7 |
| 1-5 | 2 | N/A | 1.2 | 1.3 | 1.5 |

As the results show, cold flow was not observed at ambient condition for these formulations.

### EXAMPLE 2

Various polymer matrix compositions were prepared as described below, and applied at a coat weight of 5.5 or 5.0 mg/cm² (as indicated) to a ScotchPak® 9732 backing and a release liner (e.g., a silicone- or fluoropolymer-coated polyester film). Drug flux over 9 hours was assessed in vitro using human cadaver skin (n=3):

| | | 9 h Flux | |
|---|---|---|---|
| | | (ug/cm2/h) | Flux |
| Formula | Composition | n = 3 | ratio |
| Daytrana ® | Daytrana ® Product | 28.7 | 1.00 |
| 50893 (□) | | | |
| 2-1 (▪) | 30% Methylphenidate | 32.1 | 1.12 |
| | 30% Duro-Tak ® 87-900A | | |
| | 40% Duro-Tak ® 87-9900 | | |
| | Coat Weight 5.5 mg/cm² | | |
| 2-2 (▴) | 30% Methylphenidate | 36.6 | 1.28 |
| | 35% Duro-Tak ® 87-900A | | |
| | 35% Duro-Tak ® 87-9900 | | |
| | Coat Weight 5.5 mg/cm² | | |
| 2-3 (Δ) | 35% Methylphenidate | 35.1 | 1.72 |
| | 30% Duro-Tak ® 87-900A | | |
| | 35% Duro-Tak ® 87-9900 | | |
| | Coat Weight 5.5 mg/cm² | | |
| 2-4 (∘) | 35% Methylphenidate | 37.3 | 1.30 |
| | 25% Duro-Tak ® 87-900A | | |
| | 40% Duro-Tak ® 87-9900 | | |
| | Coat Weight 5.5 mg/cm² | | |
| 2-5 () | 40% Methylphenidate | 37.4 | 1.30 |
| | 25% Duro-Tak ® 87-900A | | |
| | 35% Duro-Tak ® 87-9900 | | |
| | Coat Weight 5.0 mg/cm² | | |
| 2-6 (♦) | 40% Methylphenidate | 35.2 | 1.23 |
| | 20% Duro-Tak ® 87-900A | | |
| | 40% Duro-Tak ® 87-9900 | | |
| | Coat Weight 5.0 mg/cm² | | |

Results are shown in FIG. 2 (flux/avg. permeation rate, μg/cm²/hr). The results show that polymer matrix compositions with 30% by weight methylphenidate (or higher) achieved drug flux greater than the Daytrana® product (e.g., 30% to 40% greater). These compositions could be used to prepare systems that achieve drug delivery comparable to the Daytrana® product in a smaller size.

The peel properties of the compositions from a release liner were studied over 16 weeks at ambient conditions. The compositions were packaged in a package system comparable to that used for Daytrana.®, e.g., in an inner pouchstock material provided in an outer package in a polypropylene tray with a silica gel desiccant. Results (Avg. Peel from Release Liner (g/0.5″) (n=3) are shown below:

| | | 4 | 8 | 12 | 16 |
|---|---|---|---|---|---|
| Formula | Initial | week | week | week | week |
| 2-1 | 2.0 | 3.7 | 3.4 | N/A | 4.0 |
| 2-2 | 2.6 | 4.0 | 3.4 | N/A | 5.4 |
| 2-3 | 3.3 | 3.6 | 3.9 | N/A | 4.5 |
| 2-4 | 3.0 | 3.4 | 4.1 | N/A | 6.0 |
| 2-5 | 2.4 | 3.4 | 3.7 | N/A | 5.3 |
| 2-6 | 2.5 | 3.5 | 3.9 | N/A | 6.2 |

As the results show, cold flow was not observed at ambient condition for these formulations.

### EXAMPLE 3

A polymer matrix composition comprising 27.5% methylphenidate was prepared as described below, and applied at a coat weight of 6.0 mg/cm² to a ScotchPak® 9732 backing and a release liner (e.g., a silicone- or fluoropolymer-coated polyester film). Drug flux over 9 hours was assessed in vitro using human cadaver skin from two donors (n=3):

| | | 9 Hour | |
|---|---|---|---|
| | | Flux | Flux |
| Formula | Composition | (ug/cm2/h) | ratio |
| Daytrana ® | Daytrana ® Product | 25.4 | 1.00 |
| 3-1 | 27.5% methylphenidate | 28.2 | 1.11 |
| | 32.5% Duro-Tak ®87-900A | | |
| | 40% Duro-Tak ® 87-9900 | | |
| Daytrana ® | Daytrana ® Product | 13.9 | 1.00 |
| 3-1 | (see above) | 13.5 | 0.97 |

Peel force from the release liner remained low after up to 6 months (latest time point tested) and no cold flow was observed.

The peel properties of the compositions from a release liner were studied over 16 weeks at ambient conditions. The compositions were packaged in a package system comparable to that used for the Daytrana.® product, e.g., in an inner pouchstock material provided in an outer package in a polypropylene tray with a silica gel desiccant. Results (Avg. Peel from Release Liner (g/0.5″) (n=3) are shown below:

RL peel (g/0.5″) ( n = 3)

| | T = 1 | T = 2 | T = 3 | T = 4 | T = 5 | T = 6 |
|---|---|---|---|---|---|---|
| T = 0 | month | month | month | month | month | month |
| 6.3 | 8.7 | 8.5 | 8.6 | 8.2 | 5.6 | 10.4 |

### EXAMPLE 4

Polymer matrix compositions comprising 35% or 40% methylphenidate were prepared as described below, and applied at a coat weight of 5.5 or 6.0 mg/cm² (respectively) to a ScotchPak® 9732 backing and a release liner (e.g., a silicone- or fluoropolymer-coated polyester film). Drug flux over 9 hours was assessed in vitro using human cadaver skin from two donors (n=3):

| | | 9 hr Flux | |
|---|---|---|---|
| Formula | Composition | (ug/cm2/h) | Flux ratio |
| Daytrana ® (♦) | Daytrana ® Product | 15.4 | 1.00 |
| 4-1 (▪) | 35% Methylphenidate | 15.9 | 1.03 |
| | 65% Gelva ® 3235 | | |
| 4-2 (▴) | 40% Methylphenidate | 19.7 | 1.28 |
| | 60% Gelva ® 3235 | | |
| 4-3 () | 35% Methylphenidate | 19.9 | 1.29 |
| | 25% Duro-Tal ® 87-900A | | |
| | 40% Duro-Tak ® 87-9900 | | |
| 4-4 (□) | 40% Methylphenidate | 21.8 | 1.42 |
| | 10% Duro-Tak ® 87-900A | | |
| | 50% Duro-Tak ® 87-9900 | | |
| Daytrana ® (♦) | Daytrana ® Product | 14.9 | 1.00 |
| 4-1 (▪) | (see above) | 18.7 | 1.26 |
| 4-2 (▴) | (see above) | 24.6 | 1.65 |
| 4-3 ( [symbol] ) | (see above) | 24.6 | 1.65 |
| 4-4 () | (see above) | 26.7 | 1.79 |

FIGS. 3 and 4 show the in vitro flux data (flux/avg. permeation rate, μg/cm²/hr) for the composition of Example 4 (for each skin sample). The results show that drug flux from the polymer matrix compositions described herein is dependent on drug loading, and that polymer matrix compositions with 35% by weight methylphenidate (or higher) achieved drug flux greater than the Daytrana® product (e.g., 30% to 40% greater). These compositions could be used to prepare systems that achieve drug delivery comparable to the Daytrana® product in a smaller size.

Collectively, the in vitro flux data show that drug flux from the compositions described herein shows a drug loading (e.g., dose) dependence.

Collectively, the peel force studies show that peel force of the compositions described herein from the release liner remain low over the test periods (e.g., 4 months or 6 months). Further, no cold flow was observed during the test period;

What is claimed is:

1. A composition for the transdermal delivery of methylphenidate in the form of a flexible finite system for topical application, comprising a polymer matrix comprising methylphenidate or a pharmaceutically acceptable salt or prodrug thereof, wherein the polymer matrix comprises a non-reactive random acrylic polymer and an acrylic block copolymer.

2. The composition of claim 1, wherein the non-reactive random acrylic polymer is made from one or more monomers selected from the group consisting of methyl acrylate, methyl methacrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, and octyl acrylamide.

3. The composition of claim 2, wherein the acrylic block copolymer is made from one or more monomers selected from the group consisting of methyl acrylate, methyl methacrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, and octyl acrylamide.

4. The composition of claim 1, wherein the acrylic block copolymer comprises one or more of poly(butyl acrylate), poly(methyl methacrylate), methyl methacrylate/butyl acrylate (MMA/BA) blocks, and methyl methacrylate/butyl acrylate/alpha methyl styrene/polypropylene glycol (MMA/BA/AMS/PPG) blocks.

5. The composition of claim 1, wherein the methylphenidate is methylphenidate free base.

6. The composition of claim 1, wherein the polymer matrix comprises from about 10% to about 50% by weight methylphenidate free base.

7. The composition of claim 1, wherein the polymer matrix comprises from about 20% to about 40% by weight methylphenidate free base.

8. The composition of claim 6, wherein the polymer matrix comprises from about 10% to about 90% by weight non-reactive random acrylic polymer.

9. The composition of claim 8, wherein the polymer matrix comprises from about 30% to about 50% by weight non-reactive random acrylic polymer.

10. The composition of claim 6, wherein the polymer matrix comprises from about 1% to about 70% by weight acrylic block copolymer.

11. The composition of claim 6, wherein the polymer matrix comprises from about 10% to about 60% by weight acrylic block copolymer.

12. The composition of claim 1, wherein the composition is capable of delivering methylphenidate over a period of time of from about 6 to about 10 hours.

13. The composition of claim 1, wherein the composition delivers methylphenidate over a period of time of about 9 hours.

14. The composition of claim 1, further comprising a backing layer.

15. The composition according to claim 14, further comprising a release liner.

16. A method for the transdermal delivery of methylphenidate, comprising topically applying a composition as claimed in claim 1 to the skin or mucosa of a subject in need thereof.

17. A method of manufacturing a composition for the transdermal delivery of methylphenidate in the form of a flexible finite system for topical application, comprising forming a polymer matrix blend comprising a non-reactive random acrylic polymer, an acrylic block copolymer, and methylphenidate or pharmaceutically acceptable salt or prodrug thereof in a solvent, applying the polymer matrix blend to a support layer, and removing any remaining solvent.
